(19) United States
(12) Patent Application Publication
(10) Pub. No.: US 2018/0317793 A1
(43) Pub. Date: Nov. 8, 2018

(54) Detection of Conduction Gaps in a Pulmonary Vein

(71) Applicant: University of Exeter, Exeter Devon (GB)

(72) Inventors: John TERRY, Exeter Devon (GB); Harry GREEN, Exeter Devon (GB)

(21) Appl. No.: 15/773,735

(22) PCT Filed: Nov. 3, 2016
(86) PCT No.: PCT/GB2016/053421

§ 371 Date: May 4, 2018

(30) Foreign Application Priority Data
Nov. 4, 2015 (GB) 1519498.8

(51) Int. Cl. A61B 5/0468; A61B 5/046; A61B 5/0402; G16H 50/50

(52) U.S. Cl. CPC A61B 5/0468; A61B 5/046; A61B 5/04525; G16H 50/50; A61B 5/04028

(57) ABSTRACT

A system adapted to detect one or more conduction gaps in a pulmonary vein of a patient, the system including a device configured to receive or obtain a plurality of pulmonary vein recordings in respect of said patient, each pulmonary vein recording being representative of electrical signals detected or predicted at respective electrodes or between respective pairs of electrodes located or simulated within said pulmonary vein, a device configured to determine a respective activation time for each of said plurality of pulmonary vein recordings and generating curve data representative of said activation times, and a device configured to determine the presence of one or more conduction gaps by identifying one or more respective earliest activation times.

### FIELD OF THE INVENTION

This invention relates generally to the detection and, optionally, location of conduction gaps in a pulmonary vein of a patient and, more particularly, to a system and method adapted to detect and locate conduction gaps in a pulmonary vein of a patient for use, in, for example, in a therapeutic support system configured to assist in the treatment of atrial fibrillation (AF) by means of pulmonary vein isolation therapy. The invention also relates to a method of reconstructing pulmonary vein signals for use in the detection of conduction gaps in a pulmonary vein of a patient.

### BACKGROUND OF THE INVENTION

Atrial fibrillation (AF), the most common cardiac arrhythmia, is commonly initiated when an ectopic beat (a disturbance of normal cardiac rhythm) within the atrium, commonly originating from a small myocardial sleeve extending over the pulmonary veins, encounters a functional or anatomical obstacle, resulting in electrical re-entry. AF can frequently lead to more severe conditions including stroke, ventricular, tachycardia, and congestive heart failure.

Pulmonary vein isolation therapy is a surgical technique which attempts to isolate the pulmonary veins from the left atrium by ablating small regions of heart tissue using radio frequency ablation to form lesions. A common form of pulmonary vein isolation therapy is circumferential radio frequency ablation, in which a circular lesion is formed, surrounding the pulmonary vein and preventing the propagation of any action potential in or out of the myocardial sleeve. The ultimate objective of pulmonary vein isolation is the complete and successful electrical isolation of the left atrium and the pulmonary vein. Such electrical isolation is monitored by the use of unipolar or bipolar recordings from a lasso catheter (typically consisting of 10 or 20 electrodes) inside the pulmonary vein. As complete electrical isolation is a difficult surgical challenge, conduction gaps often remain in the lesion.

Current surgical practice requires the surgeon to study the pulmonary vein recordings from the electrodes and, by intuition and experience, attempt to determine the presence and location of a conduction gap by identifying the pulmonary vein recording in which the earliest ‘spikes’ are observed. However, this method is prone to error and can be particularly difficult when more than one conduction gap is present. Furtheromore, it is not uncommon for pulmonary vein recordings to be poor (having ‘missing’ signals) due to poor electrode contact with the patient. Clearly, such poor pulmonary vein (PV) recordings can severely reduce the accuracy of conduction gap detection. Still further, this procedure can take 30 minutes or more to complete, whereas minimising the time taken to complete surgery is an ongoing desire, not only in terms of the clinician's time, but also in view of the fact that time in the operating theatre is a known independent predictor of atrial fibrillation recurrence rate. Whilst the success rate of pulmonary vein isolation is approximately 85%, ablation of the pulmonary veins carries a risk of pulmonary vein stenosis. Furthermore, if complete electrical isolation is not achieved, the surgery can become pro-arrhythmic through the creation of conduction obstacles that facilitate the initiation of re-entrant waves. It would, therefore, be desirable to provide a system and method adapted to guide radio frequency ablation therapy by efficiently and consistently identifying conduction gaps with a view to minimising the lesions formed through ablation whilst ensuring complete pulmonary vein isolation, thereby minimising unnecessary damage to healthy atrial tissue and optimising surgery outcome.

### SUMMARY OF THE INVENTION

Aspects of the present invention seek to address at least some of these issues and, in accordance with a first aspect of the present invention, there is provided a system adapted to detect one or more conduction gaps in a pulmonary vein of a patient, the system including a device configured to receive or obtain a plurality of pulmonary vein recordings in respect of said patient, each pulmonary vein recording being representative of electrical signals detected or predicted at respective electrodes or between respective pairs of electrodes located or simulated within said pulmonary vein, a device configured to determine a respective activation time for each of said plurality of pulmonary vein recordings and generating curve data representative of said activation times, and a device configured to determine the presence of one or more conduction gaps by identifying one or more respective earliest activation times.

In an exemplary embodiment, the system may comprise a device configured to normalise said curve data to generate a relative activation time curve. The system may comprise a device configured to normalise said curve data to zero to generate a relative activation time curve. The system may further comprise a device configured to determine the location of one or more conduction gaps by finding one or more respective minima of said relative activation time curve. Alternatively, the system may comprise a device configured to determine the location of one or more conduction gaps by obtaining a weighted approximation towards an electrode having a next earliest activation time.

The system of the present invention may be configured to use real pulmonary vein recordings obtained using a lasso catheter (or similar device) located within the patient's pulmonary vein. However, the data resulting from this procedure can be noisy and may increase computational effort with regard to the detection of conduction gaps. Thus, in an exemplary embodiment of the invention, the pulmonary vein recordings may be synthetic pulmonary vein recordings, which may be less noisy than real recordings and, therefore, require less computational effort to locate conduction gaps.

In this case, the system may comprise a device adapted and configured to receive patient data, a device adapted and configured to apply said patient data to a phenomenological model representative of human ventricular action potential, and a device adapted and configured to generate said synthetic pulmonary vein recordings by applying an excitation signal to said model, and obtaining resulting output signals. One or more parameters of said phenomenological model may be fixed by a biophysical model. The biophysical model may be an atrial model, and the system may comprise a device adapted and configured to apply said patient data to said biophysical model and use parameters obtained therefrom to generate said phenomenological model. The phenomenological model may include a definition of propagation of a transmembrane voltage and the system comprises a device for numerically manipulating said definition over a substantially cylindrical domain. In an exemplary embodiment, the patient data may comprise ablation times and locations in respect of a pulmonary vein of said patient, and the system may comprise a device adapted and configured to apply one or more virtual ablations to said phenomenological model using said patient data.

The pulmonary vein recordings may be, or include, real pulmonary vein recordings obtained from said patient. In this case, in an exemplary embodiment, the system may further comprise a reconstruction module configured to:
fit a model defining said synthetic pulmonary vein recordings to said real pulmonary vein recordings;
identify any inadequate signals in said real pulmonary vein recordings; and
replace said inadequate signals with corresponding signals from said model defining said synthetic pulmonary vein recordings.

Optionally, a minimisation algorithm may be employed to fit said model defining said synthetic pulmonary vein recordings to said real pulmonary vein recordings.

In accordance with another aspect of the present invention, there is provided a computer program element comprising computer code means to make a computer execute a method of detecting one or more conduction gaps in a pulmonary vein of a patient, the method including receiving or obtaining a plurality of pulmonary vein recordings in respect of said patient, each pulmonary vein recording being representative of electrical signals detected or predicted at respective electrodes or between respective pairs of electrodes located or simulated within said pulmonary vein, determining a respective activation time for each of said plurality of pulmonary vein recordings and generating curve data representative of said activation times, and using the curve data to determine the presence and, optionally the location, of one or more conduction gaps in the pulmonary vein. In an exemplary embodiment, the method may further comprise normalising the curve data to generate a relative activation time curve, and determining the location of one or more conduction gaps by, either a) finding one or more respective minima of said relative activation time curve; or b) obtaining a weighted approximation towards an electrode having a next earliest activation time.

In accordance with yet another aspect of the present invention, there is provided a reconstruction module for a system substantially as described above, comprising a computer program element comprising computer code means to make a computer execute a method comprising the steps of:
receiving real pulmonary vein recordings obtained from said patient;
obtain synthetic pulmonary vein recordings in respect of said patient;
fit a model defining said synthetic pulmonary vein recordings to said real pulmonary vein recordings;
receive information identifying any inadequate signals in said real pulmonary vein recordings; and
replace said inadequate signals with corresponding signals from said model defining said synthetic pulmonary vein recordings.

Whilst the invention has been described above, it extends to any inventive combination of features set out above or in the following description. Although illustrative embodiments of the invention are described in detail herein with reference to the accompanying drawings, it is to be understood that the invention is not limited to these precise embodiments. As such, many modifications and variations will be apparent to practitioners skilled in the art. Furthermore, it is contemplated that a particular feature described either individually or as part of an embodiment can be combined with other individually described features, or parts of other embodiments, even if the other features and embodiments make no mention of the particular feature. Thus, the invention extends to such specific combinations not already described.

Thus, in a first exemplary embodiment, the present invention utilises a generic phenomenological model for cardiac action potential propagation that can be used to produce synthetic pulmonary vein recordings, although in other exemplary embodiments, true pulmonary vein recordings, obtained during surgery or standard clinical procedure, can also be used, and in some exemplary embodiments, such true pulmonary vein recordings may be reconstructed by replacing any ‘flat’ signals with the corresponding signals from a model of the above-mentioned synthetic pulmonary vein recordings. In either case, the present invention proposes a novel and computationally efficient method for identifying and locating one or more conduction gaps in near real time, thereby enabling the resultant system to be used as a guide during surgery, in contrast to previously proposed methods. A further advantage of at least some exemplary embodiments of the invention is that model parameters can be estimated from the data routinely collected by a cardiologist during the standard clinical procedure.

### BRIEF DESCRIPTION OF THE DRAWINGS

These and other aspects of the present invention will be apparent from the following specific description in which embodiments of the present invention are described, by way of examples only, and with reference to the accompanying drawings, in which:

FIG. 1 is a schematic flow diagram illustrating steps of a method for generating synthetic pulmonary vein recordings from patient data, for use in an exemplary embodiment of the present invention;

FIG. 2 is an illustration of simulated pulmonary vein (PV) recordings obtained using a method according to an exemplary embodiment of the present invention;

FIG. 3 is a schematic flow diagram illustrating steps of a method according to an exemplary embodiment of the present invention for the detection and location of one or more conduction gaps using synthetic or real pulmonary vein recordings;

FIG. 4 is an illustration of a relative activation time curve used in an exemplary embodiment of the present invention for the detection and location of one or more conduction gaps; and

FIG. 5 is a schematic flow chart illustrating the steps of an exemplary method of signal reconstruction for use in an exemplary embodiment of the present invention.

### DETAILED DESCRIPTION OF EMBODIMENTS

As will be known to a person skilled in the art, many phenomenological models exist that could be used to model the pulmonary vein action potential and, as such, could be utilised within the method and system proposed by aspects of the present invention. In the following specific description, reference is made to the four variable BOCF model for human ventricular action potential, as described in detail by Bueno-Orovio A, Cherry E M, Fenton F H (2008). Minimal model for human ventricular action potentials in Tissue. J Theor Biol 253: 544-560. However, it is to be understood that the present invention is not necessarily intended to be limited in this regard, and other phenomenological models, such as Fenton-Karma for example, may also be used. Indeed, it is to be understood that a monodomain or a biodomain model could be used, and examples of both will be known to a person skilled in the art.

In the above-mentioned BOCF model (and, indeed in the above-mentioned Fenton-Karma model), the propagation of the transmembrane voltage is given by:

$$\frac{\partial u}{\partial t} = ∇ . ( D ∇ u ) - ( {J}_{fi} + {J}_{so} + {J}_{si} ) \qquad ( 1 )$$

where J_{fi}, Jₛₒ and Jₛᵢ are phenomenological summations of the fast inwards, slow outwards and slow inwards currents, and D is a diffusion tensor. In this exemplary embodiment, the BOCF model is adapted through parameter estimation using the output of the detailed biophysical Courtemanche model for the human atrium as a proxy for action potential data, and the resultant biodomain model for the surface potential(I) at an electrode positioned at (x′, y′), which has been shown to reproduce atrial action potentials from atrial cells following AF-induced electrical remodelling, is given by:

$$\begin{matrix}φ ( {x}^{′} , {y}^{′} ) = aD ( {x}^{′} , {y}^{′} ) \frac{1}{r} \int ( - ∇ u ( x , y ) ) ( \frac{1}{r} ) dx & ( 2 ) \\ where r = \sqrt{{( {x}^{′} - x )}^{2} + {( {y}^{′} - y )}^{2}} & ( 3 )\end{matrix}$$

as set out in Gima K, Rudy Y (2002) Ionic current basis of electrocardiographic waveforms: a model study. Circ Res. 90 889-896.

Parameter fitting may be performed using the Nelder-Mead Simplex Algorithm, for example, by minimising the mean squared error, although many suitable methods will be apparent to a person skilled in the art and the present invention is not necessarily intended to be limited in this regard. An uneven temporal mesh can be used to perform the fit consisting of the beginning and peak of the action potential, followed by 6 evenly spaced intervals up to the APD90 to ensure a good fit for the upstroke potential. This step is taken as the emergent electrical activity is potentially constrained by the underlying structure and function of the action potential, therefore it is important to consider both the shape of the waveform as well as its conduction.

The use of the phenomenological model described above provides a pragmatic balance between the quality of the simulated signal and the computational time required to simulate the output. For example, many detailed biophysical cardiac models require a very long time to compute. In contrast, a computationally efficient model can be run multiple times for parameter estimation and sensitivity analysis over much shorter timescales.

In a first exemplary embodiment, the pulmonary vein is modelled as a cylinder by numerical integration of equation (1) over a cylindrical domain to represent the excitable myocardial sleeve extending over the base of the pulmonary vein.

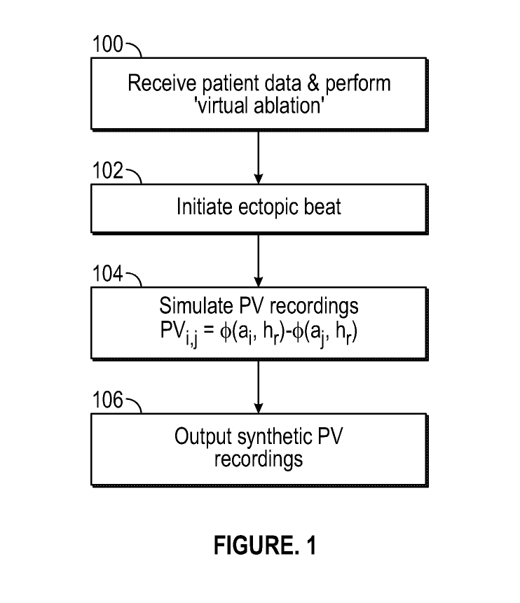

Referring to FIG. 1 of the drawings, at step 100, patient data representative of ablation times and location within the pulmonary vein is used to perform ‘virtual ablation’ in respect of the model by introducing a line of lesions on the circle y=h_{α}. At step 102, an ectopic beat is initiated from a stimulus at a random point along the line y=1, the edge of the myocardial sleeve furthest from or closest to the atrial junction, and consequently a semi-circular wavefront forms on the other side of the above-mentioned lesions. Now, at step 104, pulmonary vein recordings from the lasso catheter can be simulated by n electrodes (where n is typically 10 or 20), on y=hᵣ, where hᵣ>h_{α}. Electrodes are simulated at the points c=(α, hᵣ) where α is representative of the relative locations of the electrodes, and equation (2) is then applied at the points c to obtain the surface potential Φ, and bipolar recordings between electrodes i and j (denoted PVᵢ₋ⱼ) are simulated by:

PVᵢ₋ⱼ=ϕ(αᵢ, hᵣ)−ϕ(αⱼ, hᵣ) (4a)

In an alternative exemplary embodiment, unipolar recordings at individual electrodes i may alternatively be used, and simulated by:

PVᵢ=ϕ(αᵢ, hᵣ) (4b)

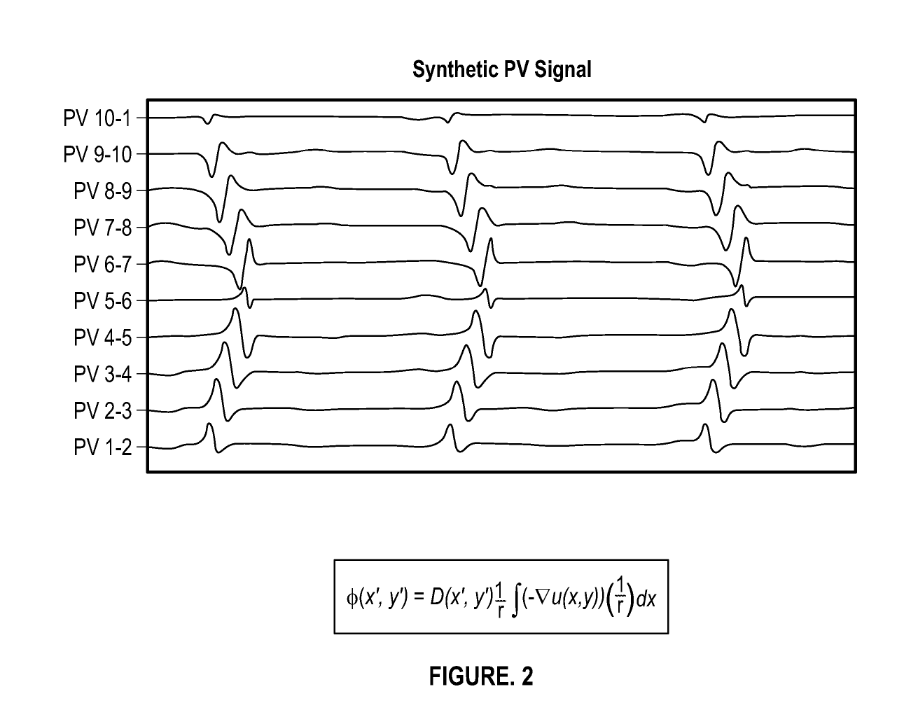

Either way, as a result, a set of synthetic pulmonary vein recording signals (one for each electrode or pair of electrodes) is output at step 106 in a format similar to the pulmonary vein recordings obtained in the conventional manner using a lasso catheter. An illustration of a set of bipolar pulmonary vein recordings (one waveform for each “channel”) is illustrated in FIG. 2 of the drawings.

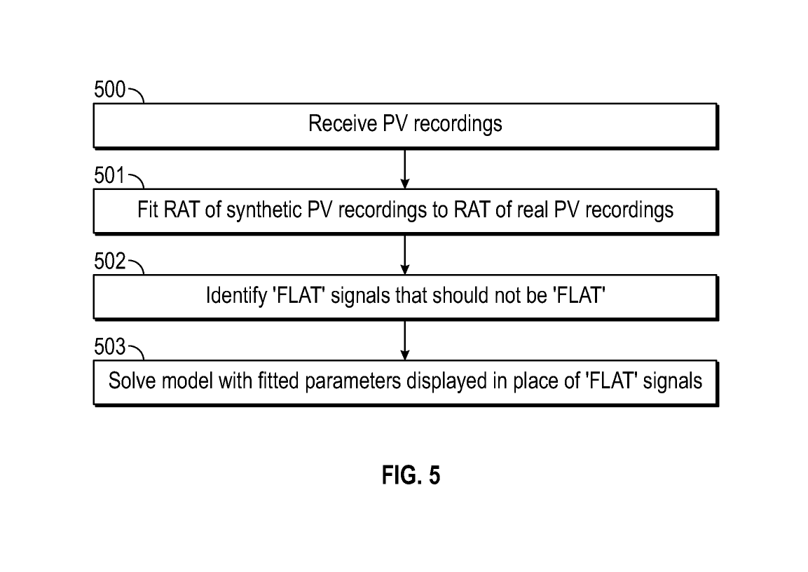

In one exemplary embodiment of the invention, these synthetic PV recordings may be used in the proposed method of conduction gap detection. However, in an alternative exemplary embodiment, real pulmonary vein recordings obtained from the patient using, for example, a lasso catheter may be used. In this case, the synthetic pulmonary vein recordings obtained in the manner described above may be used to reconstruct any ‘missing’ signals. In general, and with reference to FIG. 5 of the drawings, a minimisation algorithm is used to fit the synthetic PV recordings (or ‘model’) to real PV data. There are many such methods, such as Nelder-Mead, genetic algorithm, statistical emulators, and the algorithm can be local or global. Thus, the present invention is not necessarily intended to be limited in this regard. The chosen minimisation algorithm is used to make a Relative Activation Time Curve (RAT) of the proposed model ‘look’ like the RAT of the real PV data (but only on the adequate signals), so as to enable the inadequate signals to be reconstructed in accordance with the model. Thus, at step 500, the method obtains real PV recordings. It then fits the RAT of the synthetic PV recordings, generated using the above-described model, to the real PV recordings using a minimisation algorithms (step 502). Next, the method detects (or receives information identifying) any ‘flat’ signals in the real PV recordings that should not be flat (step 502) and, finally, solves the model with fitted signal parameters displayed instead of the ‘flat’ signals (step 503). The method may identify such ‘flat signals’ automatically, but in another exemplary embodiment, they may be visually identified by a clinician and data representative thereof (e.g. by clicking on them) used to identify them.

The following description of conduction gap detection utilised in the system and method of exemplary embodiments of the present invention is equally applicable to real pulmonary vein recordings (with or without reconstructed signals) and entirely synthetic pulmonary vein recordings obtained in the manner described above. Thus, the term “patient data” used herein may comprise the ablation time and location data required to model the pulmonary vein and, hence, generate the synthetic pulmonary vein recordings in the manner described above, or it may comprise the true pulmonary vein recordings obtained from the patient using a lasso catheter (or the like), optionally reconstructed in the manner described above.

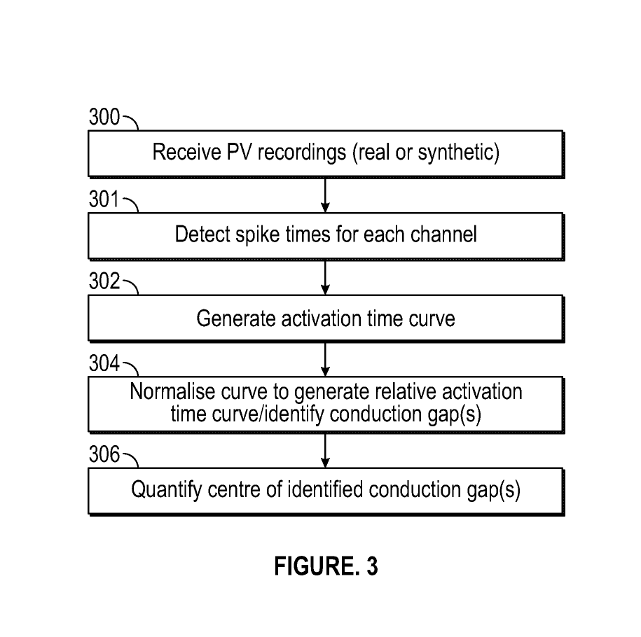

A system according to an exemplary embodiment of the present invention is adapted and configured to apply a numerical algorithm to the pulmonary vein recordings in order to detect and locate one or more conduction gaps, in a sufficiently computationally efficient manner to enable the system to be used as a guide for pulmonary vein isolation therapy during surgery. Referring to FIG. 3 of the drawings, at step 300, the method receives patient data (i.e. simulated or real PV recordings). At step 301, the ‘spike’ times are detected for each channel by finding the maximum/minimum with the greatest absolute value with a minimum threshold on the second derivative. For each ectopic beat, the electrodes closest to the conduction gap will spike first. The pattern of activation (‘spike’) times is an estimation of the above-mentioned wavefront, using the electrodes as the points of reference. Thus, at step 302, a curve representative of the activation times is generated to represent the wavefront.

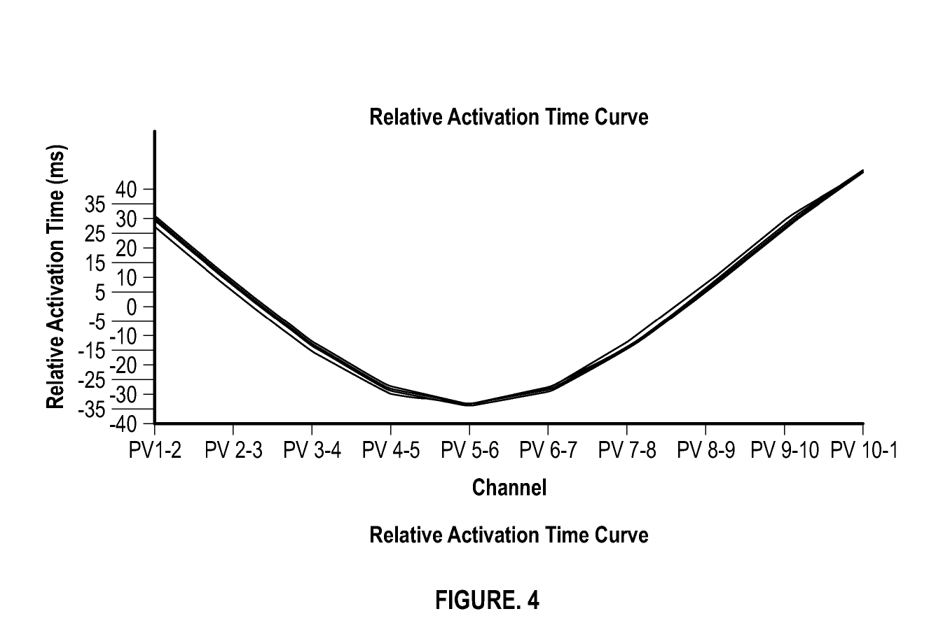

Next, at step 304, the curve is normalised such that the average is 0. As a result, each point on the curve is representative of the delay of the activation time compared to the other signals. This curve is termed herein the relative activation time curve, and an example is shown in FIG. 4 of the drawings. As an example, a value of −10 would indicate a spike time 10 ms before the average. The use of an average of relative activation time curves herein eliminates the effect of multiple onset locations and allows the detection of multiple conduction gaps, which has until now been a difficult clinical challenge.

Next, at step 306, the centre of the conduction gap(s) is quantified by one of two methods, described below, wherein the minimum of the relative activation time curve is given by PV(i).

In a first exemplary method (a “quadratic method”), the system is adapted and configured to fit a quadratic through (PV(i−1), PV(i), PV(i+1)) and find the minimum of the quadratic. In other words, the minimum of the relative activation time curve is computed.

In a second exemplary method (a “linear method”), the system is adapted and configured to take a weighted approximation towards the next earliest neighbouring electrode, i.e. if PV(i−1)<PV(i+1), the conduction gap approximation is weighted towards PV(i−1).

It will be understood by a person skilled in the art, from the foregoing description, that modifications and variations can be made to the described embodiments without departing from the scope of the invention as defined by the appended claims.

1. A system for detection of one or more conduction gaps in a pulmonary vein of a patient, the system comprising:
a first device configured to receive or obtain a plurality of pulmonary vein recordings in respect of said patient, each pulmonary vein recording being representative of electrical signals detected or predicted at respective electrodes or between respective pairs of electrodes located or simulated within said pulmonary vein,
a second device operably coupled to the first device and configured to determine a respective activation time for each of said plurality of pulmonary vein recordings and generating curve data representative of said activation times, and
a third device operably coupled to the second device and configured to determine the presence of one or more conduction gaps by identifying one or more respective earliest activation times.

2. The system according to claim 1, further comprising a device configured to normalise said curve data to generate a relative activation time curve.

3. The system according to claim 1, further comprising a device configured to normalise said curve data to zero to generate a relative activation time curve.

4. The system according to claim 3, further comprising a device configured to determine the location of one or more conduction gaps by finding one or more respective minima of said relative activation time curve.

5. The system according to claim 3, further comprising a device configured to determine the location of one or more conduction gaps by obtaining a weighted approximation towards an electrode having a next earliest activation time.

6. The system according to claim 1, wherein said pulmonary vein recordings are, or include, synthetic pulmonary vein recordings.

7. The system according to claim 6, further comprising a device adapted and configured to receive patient data, a device adapted and configured to apply said patient data to a phenomenological model representative of human ventricular action potential, and a device adapted and configured to generate said synthetic pulmonary vein recordings by applying an excitation signal to said model, and obtaining resulting output signals.

8. The system according to claim 7, wherein one or more parameters of said phenomenological model are fixed by a biophysical model.

9. The system according to claim 8, wherein said biophysical model is an atrial model, and the system comprises a device adapted and configured to apply said patient data to said biophysical model and use parameters obtained therefrom to generate said phenomenological model.

10. The system according to claim 9, wherein said phenomenological model includes a definition of propagation of a transmembrane voltage and the system comprises a device for numerically manipulating said definition over a substantially cylindrical domain.

11. The system according to claim 6, wherein said patient data comprises ablation times and locations in respect of a pulmonary vein of said patient, and the system comprises a device adapted and configured to apply one or more virtual ablations to said phenomenological model using said patient data.

12. The system according to claim 1, wherein said pulmonary vein recordings are, or include, real pulmonary vein recordings obtained from said patient.

13. The system according to claim 12, further comprising a reconstruction module configured to:
fit a model defining said synthetic pulmonary vein recordings to said real pulmonary vein recordings;
receive information identifying any inadequate signals in said real pulmonary vein recordings; and
replace said inadequate signals with corresponding signals from said model defining said synthetic pulmonary vein recordings.

14. The system according to claim 13, wherein a minimisation algorithm is employed to fit said model defining said synthetic pulmonary vein recordings to said real pulmonary vein recordings.

15. A computer program element comprising computer code to make a computer execute a method of detecting one or more conduction gaps in a pulmonary vein of a patient, the method comprising:
including receiving or obtaining a plurality of pulmonary vein recordings in respect of said patient, each pulmonary vein recording being representative of electrical signals detected or predicted at respective electrodes or between respective pairs of electrodes located or simulated within said pulmonary vein,
determining a respective activation time for each of said plurality of pulmonary vein recordings and generating curve data representative of said activation times,
normalising the curve data to generate a relative activation time curve, and
determining the location of one or more conduction gaps by, either a) finding one or more respective minima of said relative activation time curve; orb) obtaining a weighted approximation towards an electrode having a next earliest activation time.

16. A reconstruction module for a system according to claim 1, comprising a computer program element comprising computer code to make a computer execute a method comprising the steps of:
receiving real pulmonary vein recordings obtained from said patient;
obtain synthetic pulmonary vein recordings in respect of said patient;
fit a model defining said synthetic pulmonary vein recordings to said real pulmonary vein recordings;
identify any inadequate signals in said real pulmonary vein recordings; and
replace said inadequate signals with corresponding signals from said model defining said synthetic pulmonary vein recordings.
